(19) United States
(12) Patent Application Publication
(10) Pub. No.: US 2025/0114080 A1
(43) Pub. Date: Apr. 10, 2025

(54) SPECIMEN COLLECTION VESSEL SYSTEM AND METHOD

(71) Applicant: Brenda Veselka Joseph, Austin, TX (US)

(72) Inventor: Brenda Veselka Joseph, Austin, TX (US)

(21) Appl. No.: 18/910,498

(22) Filed: Oct. 9, 2024

Related U.S. Application Data
(60) Provisional application No. 63/543,126, filed on Oct. 9, 2023.

(51) Int. Cl. A61B 10/00

(52) U.S. Cl. CPC A61B 10/007

(57) ABSTRACT

A system for specimen collection for use in urine collection includes an extended cylinder designed to fit at least partially within a standard urine specimen cup and an outwardly tapering wall that forms a broad collection area. The outwardly tapering wall may include an ergonomic rim with a tear-drop shaped perimeter to ensure a comfortable and secure fit during use. The vessel reduces the risk of spillage and contamination, enhancing the accuracy and hygiene of sample collection. The vessel may be provided in both disposable and reusable forms, depending on the intended use case. The specimen collection vessel is particularly beneficial for female users, children, and elderly individuals.

### TECHNICAL FIELD

The present disclosure relates generally to lab and medical testing, and, more particularly, relates to urine collection devices and uses.

### BACKGROUND

Urine collection is a routine practice in medical diagnostics that presents several challenges, especially for female users, children, and elderly individuals. Traditionally, urine is collected in specimen cups, which are simple cylindrical containers with a removable lid. For female, elderly, children and other users, aligning the specimen cup to capture the urine stream is often difficult and can result in spills, discomfort, and sample contamination. Furthermore, the lack of an appropriate interface between the user's anatomy and the collection vessel exacerbates these challenges, resulting in sanitation issues and reduced accuracy of sample collection.

It would therefore be advantageous to provide a specimen collection vessel that facilitates accurate, comfortable, and hygienic urine collection. It would further be advantageous to ensure compatibility with different sizes of existing specimen cups used in healthcare environments.

### SUMMARY

The present invention addresses these challenges and others by providing an ergonomic urine collection vessel that integrates with standard specimen cups, providing a ready fit and reducing spillage, contamination, and discomfort during use.

An embodiment of the invention is a system for specimen collection, for use with a urine specimen cup having an opening, including an extended cylinder configured to fit at least partially within the opening of the urine specimen cup, and an outwardly tapering wall extending from the extended cylinder to form a broad collection opening.

Another embodiment of the invention is a method for urine collection, the method includes obtaining an extended cylinder connected to an outwardly tapering wall of a funnel structure, the extent of the outwardly tapering wall forms a somewhat tear drop shape, fitting the extended cylinder at least partially into a specimen cup, placing the tear drop shape at least partially around the genitals, receiving urine into the funnel structure, and guiding by the funnel structure the urine into the specimen cup.

Yet another embodiment of the invention is a method of manufacture, the method of manufacture includes forming an extended cylinder, forming a tapering wall connected to the extended cylinder, and forming an extent of the tapering wall opposite the extended cylinder, in a tear-drop shape.

### BRIEF DESCRIPTION OF THE DRAWINGS

The present invention is illustrated by way of example and not limitation in the accompanying figures, in which like references indicate similar elements, and in which:

FIG. 1 illustrates a top and right-side perspective view of a system for collecting a specimen, according to certain embodiments of the invention;

FIG. 2 illustrates a bottom and right-side perspective view of the system of FIG. 1, according to certain embodiments of the invention;

FIG. 3 illustrates the system of FIGS. 1 and 2 positioned above a standard urine specimen cup, according to certain embodiments of the invention;

FIG. 4A illustrates a right-side view of the system of FIGS. 1 and 2, indicating how it fits with specimen cups of smaller sizes (e.g., 2 oz.), according to certain embodiments of the invention;

FIG. 4B illustrates a right-side view of the system of FIGS. 1 and 2, indicating how it fits with specimen cups of larger sizes (e.g., 4 oz.), according to certain embodiments of the invention;

FIG. 5 illustrates a top view of the system of FIGS. 1 and 2, with grip-enhancing features on the rim for improved handling and flow-guiding channels on an inner surface of a tapering wall, according to certain embodiments of the invention; and

FIG. 6 illustrates a method of operation of a system for collecting a specimen, according to certain embodiments of the invention.

### DETAILED DESCRIPTION

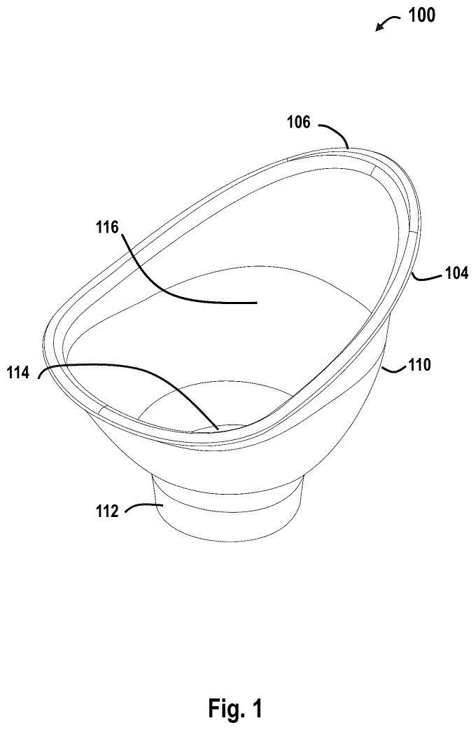

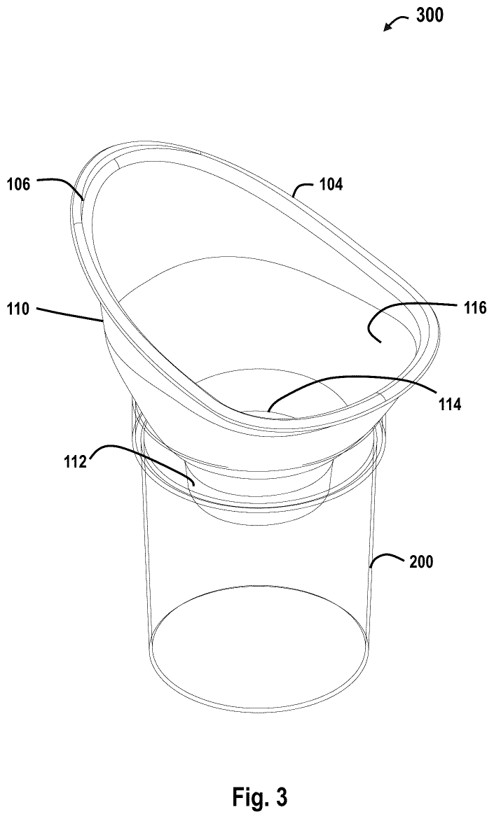

Referring to FIGS. 1, 2 and 3, in conjunction, a system 100, 200, 300 includes an extended cylinder 112 and an outwardly tapering wall 110. The extended cylinder 112 is dimensioned to fit at least somewhat within a urine specimen cup 200. This fit prevents unintended movement during urine collection, ensuring that the specimen cup 200 remains stable throughout the process.

The outwardly tapering wall 110 extends from an upper portion 114 of the extended cylinder 112 and diverges outward to form a sloping funnel-like structure 116, guiding the urine directly into the specimen cup 200. The tapering wall 110 reduces splashing and makes the collection process more hygienic by minimizing exposure of the user's hands to the collected specimen.

At the top of the tapering wall 110 is an ergonomic rim 106, forming a tear-drop shaped perimeter 104 that conforms comfortably to genitalia, particularly but nonexclusively that of a female. This tear-drop shaped perimeter 104 and tapering wall 110 provides somewhat of a seal that prevents spillage and enhances comfort, making the collection process easier and more reliable for female users, as well as for children and elderly individuals who may find it difficult to aim accurately.

The rim 106 may also include grip-enhancing textures, enabling better control during positioning and use.

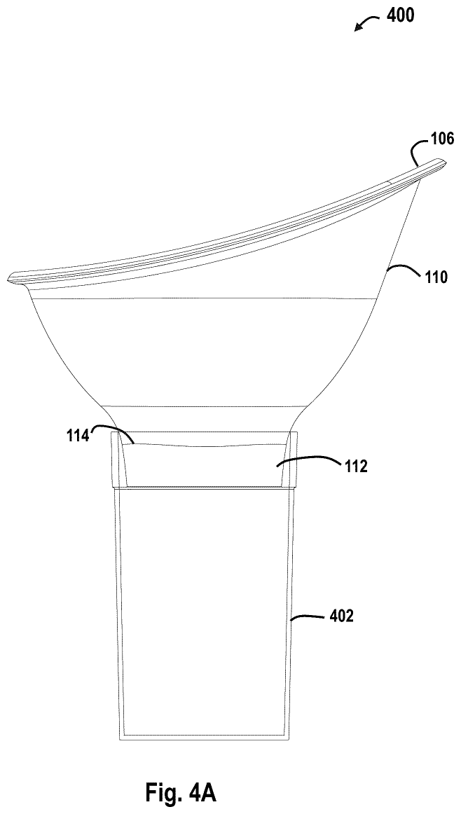

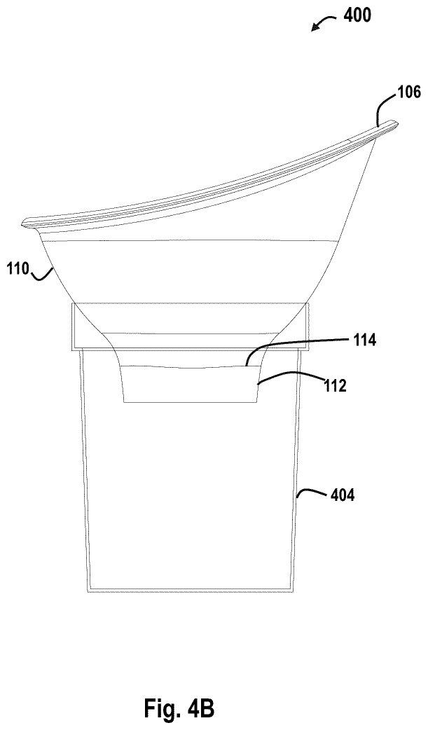

Referring to FIGS. 4A and 4B, a system 100,200,300 such as that of FIGS. 1-3, is mated with a smaller specimen cup 402, such as for nonexclusive example a 2 oz. cup, and is mated with a larger specimen cup 404, such as for nonexclusive example a 4 oz. cup, respectively. In each instance, the extended cylinder 112 extends at least somewhat into the cup 402, 404. Additionally, the graduated sloping funnel-like structure 116 of the outwardly tapering wall 110 can conform to the opening of the cup 402, 404 for larger opening. This prevents spillage in use.

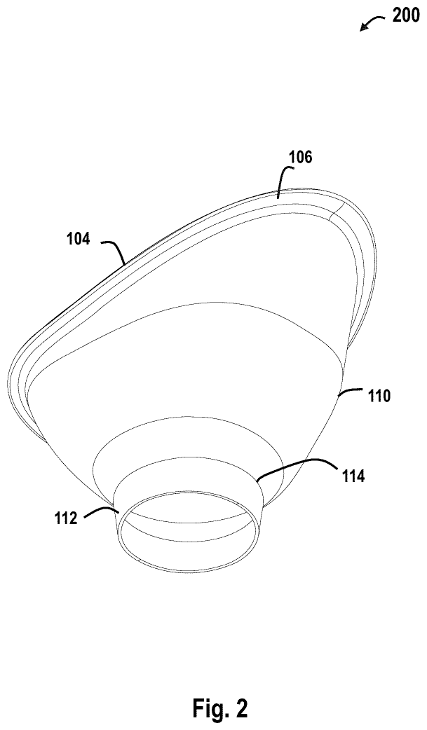

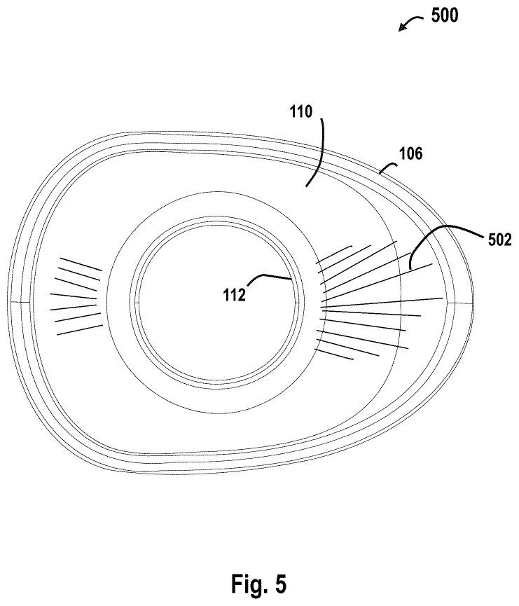

Referring to FIG. 5, in certain nonexclusive embodiments, a system 500, such as that of FIGS. 1 and 2, includes flow-guiding channels 502 integrated into the inner surface 504 of the tapering wall 110. These channels 502 are designed to direct urine smoothly into the extended cylinder 112, thereby reducing splashing and ensuring that the entire sample is collected efficiently.

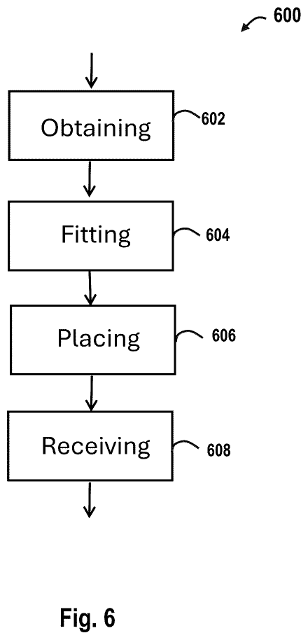

Referring to FIG. 6, a method 600 of specimen collection includes obtaining 602 a system 100, 200 formed with an extended cylinder and outwardly tapering wall of a funnel structure, the extent of the outwardly tapering wall forms a tear drop shape. Fitting 604 the extended cylinder at least partially into a specimen cup. Placing 606 the tear drop shape at least partially around the genitals. Receiving 608 urine into the funnel structure. Guiding 610 by the funnel structure the urine into the specimen cup.

In various alternatives, the systems and devices may be provided in either disposable or reusable forms. The disposable version is intended for single use in clinical environments to prevent cross-contamination, while the reusable version is constructed from a medical-grade material that can be sterilized, providing a sustainable option for personal use.

### Example Use Cases

Clinical Settings: In clinical environments, the specimen collection vessel can be provided to patients who require urine testing. Its ergonomic design ensures that patients, particularly female patients, are able to use it comfortably without the risk of spillage or contamination, making it easier for healthcare professionals to obtain accurate samples.

Home Use: The reusable version of the vessel can be part of a home diagnostic kit. Patients can easily collect samples themselves and transfer them to healthcare providers for further analysis.

Geriatric and Pediatric Use: For elderly users and children, aligning a traditional specimen cup can be challenging. The ergonomic rim and broad collection surface of the vessel provide an effective solution, making the process accessible and comfortable.

In other alternatives, the specimen collection vessel system and method embodiments may include particular formations for guiding urine flow in the system and method, particular materials for reusable or extended use, and other variations.

In the foregoing specification, the invention has been described with reference to specific embodiments. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention.

Benefits, other advantages, and solutions to problems have been described above with regard to specific embodiments. However, the benefits, advantages, solutions to problems and device(s), connection(s) and element(s) that may cause any benefit, advantage, or solution to occur or become more pronounced are not to be construed as a critical, required, or essential feature or element of any or all the claims. As used herein, the terms “comprises, “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A system for specimen collection, for use with a urine specimen cup having an opening, comprising:
an extended cylinder configured to fit at least partially within the opening of the urine specimen cup;
an outwardly tapering wall extending from the extended cylinder to form a broad collection opening.

2. The system of claim 1, further comprising:
a rim with a somewhat tear-drop shaped perimeter at a top of the wall opposing the extended cylinder, wherein the rim and perimeter are configured to comfortably fit against genitalia, minimizing spillage.

3. The system of claim 1, wherein the extended cylinder is configured to fit urine specimen cups of multiple sizes selected from the group of: 2 oz. cup, 4 oz. cup, and combinations of these.

4. The system of claim 1, wherein the system is constructed from materials suitable for sterilization.

5. The system of claim 1, wherein the system is suitable for use selected from the group of: repeated use, disposable, and combinations of these.

6. The system of claim 2, further comprising grip-enhancing features on the rim to assist users in handling the system during collection.

7. They system of claim 1, wherein the outwardly tapering wall includes an inner surface, and further comprising:
flow-guiding channels on the inner surface of the outwardly tapering wall, configured to direct urine smoothly into the extended cylinder, reducing splashing.

8. A method for urine collection, comprising:
obtaining an extended cylinder connected to an outwardly tapering wall of a funnel structure, the extent of the outwardly tapering wall forms a somewhat tear drop shape;
fitting the extended cylinder at least partially into a specimen cup;
placing the tear drop shape at least partially around the genitals;
receiving urine into the funnel structure; and
guiding by the funnel structure the urine into the specimen cup.

9. The method of claim 8, further comprising:
funneling urine of receiving by guide channels of an inner surface of the outwardly tapering wall of the funnel structure.

10. A method of manufacture, comprising:
forming an extended cylinder;
forming a tapering wall connected to the extended cylinder; and
forming an extent of the tapering wall opposite the extended cylinder, in a tear-drop shape.

11. The method of manufacture of claim 10, further comprising:
forming a rim of the extent of the tapering wall.

12. The method of manufacture of claim 10, further comprising:
forming the tapering wall with an inner surface of guide structures.

13. The method of manufacture of claim 11, further comprising:
forming the tapering wall with an inner surface of guide structures.

14. The method of manufacture of claim 10, wherein each forming is performed by moulding.

15. The method of manufacture of claim 10, wherein each forming is performed by extrusion.
